(19) United States
(12) Patent Application Publication
(10) Pub. No.: US 2026/0056212 A1
(43) Pub. Date: Feb. 26, 2026

(54) METHOD FOR MEASURING MINERALOCORTICOID RECEPTOR ACTIVITY

(71) Applicant: OSAKA UNIVERSITY, Osaka (JP)

(72) Inventors: Tomoaki HAYAKAWA, Osaka (JP); Atsunori FUKUHARA, Osaka (JP); Iichiro SHIMOMURA, Osaka (JP)

(73) Assignee: OSAKA UNIVERSITY, Osaka (JP)

(21) Appl. No.: 18/877,763

(22) PCT Filed: Jun. 14, 2023
(86) PCT No.: PCT/JP2023/022024

§ 371 Date: Dec. 20, 2024

(30) Foreign Application Priority Data
Jun. 30, 2022 (JP) 2022-106278

(51) Int. Cl. G01N 33/68; G01N 33/50; G01N 33/74

(52) U.S. Cl. CPC G01N 33/6893; G01N 33/5091; G01N 33/743; G01N 2333/70596; G01N 2333/723; G01N 2800/042; G01N 2800/044; G01N 2800/32; G01N 2800/50; G01N 2800/60; G01N 2800/7066

(57) ABSTRACT

[Problem] Various pathological conditions cause a mineralocorticoid receptor (MR) to be activated, and excessive activation of the MR is a risk factor of onset of cerebrovascular and cardiovascular diseases. However, no indicator has provided direct assessment of MR activity, and hence early detection of excessive MR activation has remained difficult. An object of the present invention is to find an indicator that can directly assess MR activity.

[Solution] It has been found that MR activity can be directly assessed by using, as an indicator, a protein quantity of an epithelial sodium channel (ENaC) subunit in urinary extracellular vesicles. The indicator is preferably a ratio of a protein quantity of an ENaC subunit to a protein quantity of an internal control protein in the extracellular vesicles. A specific aspect is a ratio of y subunit of ENAC to CD9 (γENaC/CD9). A test is performed for a disease associated with MR activation to allow an early start of treatment. The early start of treatment enables prevention of onset of cerebrovascular and cardiovascular diseases.

### TECHNICAL FIELD

The present invention relates to a method of measuring mineralocorticoid receptor activity.

### BACKGROUND ART

Primary aldosteronism (hereinafter also referred to as “PA”) is a disease that exhibits hypertension due to autonomous hypersecretion of aldosterone from an adrenal grand and appears in approximately 10% of hypertension patients. Aldosterone activates a mineralocorticoid receptor (hereinafter also referred to as “MR”) and elicits hypertension. PA is known to be complicated by cerebrovascular and cardiovascular diseases, such as stroke or atrial fibrillation, with higher frequency as compared to essential hypertension in cases identical in age, sex, and blood pressure (Non Patent Literature 1). However, there is no linear correlation between the blood concentration of aldosterone and the risk of cerebrovascular and cardiovascular diseases. This is considered because excess intake of salt, hyperglycemia, obesity, or the like may be involved in activation of the MR (Non Patent Literatures 2 and 3). Furthermore, several recent epidemiological studies for PA have revealed that MR activation is a risk factor of cerebrovascular and cardiovascular diseases (Non Patent Literatures 4 and 5). A cardiovascular disease is the second leading cause of death and a cerebrovascular disease is the fourth in Japan, indicating the significance of management of their risk factors. MR activity is estimated from several kinds of data, such as blood potassium concentration or blood renin activity, but these are affected by many factors other than MR activity, and there has been to date no indicator that enables direct assessment of MR activity.

Although PA has been treated with MR inhibitors, MR inhibitors do not reduce the blood concentration of aldosterone, thus preventing use of blood aldosterone concentration as an indicator for determining a therapeutic effect. Thus, there has been a need for a better marker for determining a therapeutic effect.

In recent years, a concept of “MR-associated hypertension” has been proposed. “MR-associated hypertension” refers to a group of hypertension effectively responsive to a MR inhibitor, and has been proposed as a pathology that involves excessive MR activation induced by hypersecretion of aldosterone, obesity, diabetes, excess intake of salt, or the like and has a high risk of onset of cerebrovascular and cardiovascular disorders (Non Patent Literature 6).

A urinary extracellular vesicle is a vesicle secreted from a tubular cell and released in urine, contains proteins, miRNAs, mRNAs, and the like, and has been recently reported to have availability as a biomarker. A report has shown that healthy subjects infused continuously with aldosterone had an increased amount of y subunit protein of the epithelial sodium channel (hereinafter also referred to as “γENaC”) in urinary extracellular vesicles (Non Patent Literature 7). In another report, a change in endocrine signal such as MR activity is accompanied by a change in mRNA in a urine supernatant (Non Patent Literature 8).

### CITATION LIST

### Non Patent Literature

[NPL 1] Monticone S, et al. Lancet Diabetes and Endocrinology 2018; 6 41-50

[NPL 2] Ohno Y, et al. Hypertension. 2018; 71:530-537

[NPL 3] Hayashi T, et al. International Heart Journal 2017; 58 794-802

[NPL 4] Savard S, et al. Hypertension. 2013; 62:331-336

[NPL 5] Mulatero P, et al. J Clin Endocrinol Metab 2013; 98:4826-4833

[NPL 6] Shibata H & Itoh H American Journal of Hypertension 2012; 25 514-523

[NPL 7] Qi Y, et al. Journal of the American Society of Nephrology 2016; 27 646-656

[NPL 8] Bazzell BG, et al. Circ Genom Precis Med. 2018; 11(9): e002213

### SUMMARY OF INVENTION

### Technical Problem

Various pathological conditions cause a mineralocorticoid receptor (MR) to be activated, and excessive activation of the MR is a risk factor of onset of cerebrovascular and cardiovascular diseases. However, no indicator has provided direct assessment of MR activity, and hence early detection of excessive MR activation has remained difficult. An object of the present invention is to find an indicator that can directly assess MR activity.

### Solution to Problem

The inventors of the present invention have made investigations in order to achieve the above-mentioned object and completed the present invention. That is, the present invention includes the following aspects.
1. A method for measuring mineralocorticoid receptor activity using an indicator, wherein the indicator is a protein quantity of an epithelial sodium channel subunit in urinary extracellular vesicles.
2. The method according to the above-mentioned item 1, wherein the indicator is a ratio of a protein quantity of an epithelial sodium channel subunit to a protein quantity of an internal control protein in extracellular vesicles.
3. The method according to the above-mentioned item 2, wherein the internal control protein in the extracellular vesicles is a tetraspanin.
4. The method according to the above-mentioned item 3, wherein the tetraspanin is CD9.
5. The method according to any one of the above-mentioned items 1 to 4, wherein the subunit comprises at least one selected from the group consisting of an α subunit, a β subunit and a γ subunit.
6. The method according to the above-mentioned item 5, wherein the subunit is the y subunit.
7. The method according to the above-mentioned item 1, wherein the method comprises using, as a sample, urine of a subject to be tested having or suspected to have at least one disease or symptom selected from the group consisting of hypertension, primary aldosteronism, diabetes, obesity and a renal disease.
8. The method according to the above-mentioned item 1, wherein the mineralocorticoid receptor activity is mineralocorticoid receptor activity in a tubular epithelial cell.
9. A method for testing a disease associated with mineralocorticoid receptor activation comprising the method of the above-mentioned item 1.
10. The method according to the above-mentioned item 9, wherein the disease associated with mineralocorticoid receptor activation is mineralocorticoid receptor-associated hypertension.
11. A method for testing a risk of onset of cerebrovascular and cardiovascular diseases comprising the method of the above-mentioned item 1.
12. The method according to the above-mentioned item 11, wherein the cerebrovascular and cardiovascular diseases comprise an arteriosclerotic disease.
13. A method for determining a therapeutic effect on a disease associated with mineralocorticoid receptor activation comprising the method of the above-mentioned item 1.
14. The method according to the above-mentioned item 13, wherein the disease associated with mineralocorticoid receptor activation is primary aldosteronism.
15. A method for testing blood aldosterone concentration comprising the method of the above-mentioned item 1.

### Advantageous Effects of Invention

According to the method of the present invention, a patient with excessive MR activation can be detected early and conveniently. In the patient with excessive MR activation, treatment for suppressing MR activity such as pharmacotherapy with a MR inhibitor can be started early. Excessive MR activation causes arteriosclerosis and elevated blood pressure, and increases the risk of onset of cerebrovascular and cardiovascular diseases, such as stroke, a coronary artery disease, atrial fibrillation, and heart failure. Early detection of excessive MR activation and early start of treatment can alleviate pathological conditions, such as arteriosclerosis and elevated blood pressure, or suppress exacerbation of the pathological conditions. Early start of treatment can also suppress onset of cerebrovascular and cardiovascular diseases such as stroke.

The method of the present invention can also be used to determine an effect of treatment for suppressing MR activity.

Patients with hypertension or a tendency to high blood pressure include patients having MR-associated hypertension with excessive MR activation. However, the current first-line drugs for pharmacotherapy of hypertension are ACE inhibitors, ARBs, Ca antagonists, and diuretics, and at present, only after resistance to these drugs is revealed, MR inhibitors become candidates for therapeutic drugs. When assessment of MR activity is made possible, early diagnosis of a patient having MR-associated hypertension and start of treatment for suppressing MR activity can be achieved. The measuring method of the present invention enables personalized medicine for hypertension.

Patients with diabetes, obesity, a renal disease, primary aldosteronism and the like include patients with excessive MR activation. Measurement of MR activity in a patient having or suspected to have any of those diseases or symptoms allows start of treatment for suppressing MR activity.

The measuring method of the present invention, which uses urine as a sample, is non-invasive, and hence provides no burden on a subject. The measuring method of the present invention, which is also applicable to a common medical examination, is quite useful because there are many subjects suspected to have obesity, hypertension, or diabetes.

The MR is a receptor for a ligand such as aldosterone. There is a linear correlation between a measured value obtained by the method of the present invention and blood aldosterone concentration, and hence the method of present invention can be used to examine blood aldosterone concentration in a subject who has received no pharmacotherapy with a MR inhibitor. When a urine test allows examination of blood aldosterone concentration, constraints to a patient are reduced such as blood collection in the morning and after rest in the supine position for 30 minutes, which is a recommended condition for measurement of aldosterone.

### BRIEF DESCRIPTION OF DRAWINGS

FIGS. 1A, 1B, 1C, and 1D show representative Western blot images of proteins in urinary extracellular vesicles (hereinafter also referred to as “uEVs”). Ur: urine; Sup: supernatant after ultracentrifugation; x: fold concentration from urine samples.

FIG. 2 shows a relationship between urine creatinine concentration and a protein quantity of CD9 in uEVs. The protein quantity of CD9 in uEVs was well correlated with the urine creatinine concentration. The values were analyzed with single linear regression. Solid circles: male patients; Open circles: female patients.

FIG. 3 shows a relationship between plasma aldosterone concentration (hereinafter also referred to as “PAC”) and a ratio of a protein quantity of γENaC to a protein quantity of CD9 in uEVs (hereinafter also referred to as “γENaC/CD9”). γENaC/CD9 was fairly strongly correlated with PAC. The values were analyzed with single linear regression. Solid circle: male patients; Open circles: female patients.

FIG. 4 shows a relationship between plasma aldosterone concentration (PAC) and a ratio of a protein quantity of NCC (NaCl cotransporter) to a protein quantity of CD9 in uEVs (hereinafter also referred to as NCC/CD9). NCC/CD9 was correlated with PAC. The values were analyzed with single linear regression. Solid circles: male patients; Open circles: female patients.

FIG. 5 shows a relationship between plasma aldosterone concentration (PAC) and serum potassium concentration. PAC and serum potassium concentration exhibited a weak correlation. The values were analyzed with single linear regression. Solid circles: male patients; Open circles: female patients.

FIG. 6 shows comparison of γENaC/CD9 in uEVs between primary aldosteronism patients having normal plasma aldosterone concentration (nPA) and primary aldosteronism patients having high plasma aldosterone concentration (hPA). The hPA group exhibited significantly higher γENaC/CD9 in uEVs than that of the nPA group. The values were analyzed with the Wilcoxon test. Bars represent mean+standard error. nPA: primary aldosteronism with normal plasma aldosterone concentration; hPA: primary aldosteronism with high plasma aldosterone concentration.

FIG. 7 shows changes in γENaC/CD9 in uEVs after PA treatment. Patient A and patient B presented marked reductions in γENaC/CD9 in uEVs through treatment with spironolactone, as well as low values after adrenalectomy. Spironolactone had only a small effect on plasma aldosterone concentration due to autonomous production of aldosterone. After adrenalectomy, neither of the two patients received MR antagonists nor potassium supplementation. Solid circles: γENaC/CD9 in uEVs; Solid squares: plasma aldosterone concentration (PAC); CCB: calcium channel blocker; Spiro: spironolactone; Adx: adrenalectomy.

### DESCRIPTION OF EMBODIMENTS

A mineralocorticoid receptor (MR) is expressed in many tissues, such as kidney, large bowel, blood vessel, heart, and central nervous system, and is activated by a mineralocorticoid such as aldosterone and a glucocorticoid such as cortisol. In the kidney, aldosterone activates the MR in a tubular epithelial cell, and leads to increases in epithelial sodium channel (hereinafter also referred to as “ENaC”), sodium-chloride cotransporter (hereinafter also referred to as NCC) and the like on a renal tubular side.

A urinary extracellular vesicle is an extracellular vesicle in urine. An extracellular vesicle in the present invention encompasses all membrane vesicles secreted from a cell, including exosomes, microvesicles (MVs), and apoptotic bodies. The extracellular vesicle in the present invention is preferably an exosome and a microvesicle, more preferably an exosome.

The method for measuring MR activity of the present invention comprises using an indicator, wherein the indicator is a protein quantity of an epithelial sodium channel (ENaC) subunit in urinary extracellular vesicles. The epithelial sodium channel (ENaC) is a heterotrimer formed of three subunits α, β and γ. ENaC participates in reabsorption of sodium ions in tubules and collecting ducts in the kidney. The epithelial sodium channel (ENaC) subunit in a urinary extracellular vesicle, a subject to be measured in the present invention, comprises at least one selected from the group consisting of an α subunit (QENaC), a β subunit (BENaC) and a ϵ subunit (γENaC). The subunit may be an inactive form or an active form. The subunit may be a partial protein or a partial peptide having a function of the subunit. A preferred subject to be measured in the present invention is a γ subunit (γENaC) and more preferably an active γ subunit.

The measuring method of the present invention which comprises using, as an indicator, a protein quantity of an ENaC subunit in urinary extracellular vesicles, has the advantages of using a more stable subject to be measured and allowing a reduction in measuring time and simplification of a measuring method, as compared to a measuring method including using mRNA or miRNA as an indicator.

The indicator in the method of the present invention is not limited as long as the indicator indicates a protein quantity of an ENaC subunit in urinary extracellular vesicles. A preferred indicator is, for example, a ratio of a protein quantity of an ENaC subunit to a protein quantity of an internal control protein in the extracellular vesicles.

The internal control protein is not particularly limited. Examples thereof include Alix, Tsg101 and tetraspanins which belong to the transmembrane protein family. A preferred internal control protein is tetraspanins, and the tetraspanins include CD63, CD81 and CD9. A preferred tetraspanin is CD9.

A preferred indicator in the method of the present invention is a protein quantity of γENaC in urinary extracellular vesicles. A more preferred indicator is a ratio of a protein quantity of γENaC to a protein quantity of an internal control protein in urinary extracellular vesicles. A more preferred indicator is a ratio of a protein quantity of γENaC to a protein quantity of a tetraspanin in urinary extracellular vesicles. An even more preferred indicator is a ratio of a protein quantity of γENaC to a protein quantity of CD9 in urinary extracellular vesicle (hereinafter also referred to as “γENaC/CD9”). γENaC/CD9 has a strong correlation with blood aldosterone concentration.

The mineralocorticoid receptor (MR) is expressed in many tissues, such as kidney, large bowel, blood vessel, heart, and central nervous system. Aldosterone and the like, which activate the MR, act on the whole body tissues, and MR activity that can be measured in the present invention may be MR activity in these tissues. In the measuring method of the present invention, a protein quantity of ENAC in urinary extracellular vesicles is measured, and hence MR activity in the kidney, particularly in a tubular epithelial cell may be directly measured.

The present application also relates to an invention of a method for testing a disease associated with MR activation, comprising the measuring method of the present invention described above. The disease associated with MR activation is a disease having a pathological condition in which the MR is excessively activated in any one of tissues or plurality of tissues in the body. The MR is excessively activated by excessive secretion of aldosterone, excessive intake of salt, diabetes, obesity or the like. Aldosterone and the like affect the whole body tissues, and hence the MRs may be activated in various tissues and lead to onset of a disease associated with MR activation or development of a symptom thereof. There is a concept of MR-associated hypertension, which is hypertension effectively responsive to a MR inhibitor. The MR-associated hypertension is one of the diseases associated with MR activation in the present invention. Primary aldosteronism having high MR activity is a disease included in the MR-associated hypertension. In a patient diagnosed to have a disease associated with MR activation, early start of treatment for suppressing MR activity, for example, pharmacotherapy such as administration of a MR inhibitor, can alleviate a symptom or suppress progression of a symptom.

The present application also relates to an invention of a method for determining a therapeutic effect on a disease associated with MR activation, comprising the measuring method of the present invention described above. In follow-up of treatment for suppressing MR activity in a patient having a disease associated with MR activation, the method of the present invention can be used for determining an effect of the treatment. The disease associated with MR activation may be, for example, MR-associated hypertension. A disease included in the MR-associated hypertension may be, for example, primary aldosteronism with high MR activity. Administration of a MR inhibitor to a patient having primary aldosteronism with high MR activity reduces MR activity, but does not necessarily reduce blood aldosterone concentration. Aldosterone concentration could not be used as an indicator for determining a therapeutic effect by administration of a MR inhibitor for primary aldosteronism, and hence there has been demand for a better indicator. To this, the present invention has enabled determination of a therapeutic effect.

Patients with hypertension or a tendency to high blood pressure include patients having MR-associated hypertension with excessive MR activation. However, the current first-line drugs for pharmacotherapy of hypertension are ACE inhibitors, ARBs, Ca antagonists, and diuretics, and at present, only after resistance to these drugs is revealed, treatment with a MR inhibitor becomes one of options. When assessment of MR activity is made possible, early diagnosis of a patient having MR-associated hypertension and start of treatment for suppressing MR activity can be achieved. The measuring method of the present invention enables personalized medicine for hypertension.

The present application also relates to an invention of a method for testing the risk of onset of cerebrovascular and cardiovascular diseases, comprising the measuring method of the present invention described above. The cerebrovascular and cardiovascular diseases encompass a cardiovascular disease and a cerebrovascular disorder, more particularly an arteriosclerotic disease, stroke, a coronary artery disease, atrial fibrillation, and heart failure. MR activation is a risk factor of cerebrovascular and cardiovascular diseases, and when MR activity is high, early start of treatment for suppressing MR activity allows prevention of onset of cerebrovascular and cardiovascular diseases. The cerebrovascular and cardiovascular diseases are the leading causes of death, and prevention of their onset is important.

A subject to be measured for MR activity is not particularly limited. Preferred subjects to be measured for MR activity include a human and non-human animals such as pets or domestic animals. When the subject to be measured for MR activity is a human, the subject may be a healthy subject or a patient having any disease or symptom. Excessive MR activation is induced by diabetes, obesity, a renal disease, primary aldosteronism, or the like, and hence a patient having or suspected to have such a disease or symptom is preferred as the subject to be measured for MR activity. In addition, the excessive MR activation causes hypertension, and thus a patient having hypertension or relatively high blood pressure is preferred as the subject to be measured for MR activity. Once elevated MR activity is revealed, treatment for suppressing MR activity can be started to alleviate the condition, pathological condition and symptom or to suppress exacerbation thereof, and also to prevent onset of cerebrovascular and cardiovascular diseases.

A sample of the measuring method of the present invention is urine from a subject to be tested. A preferred sample is, for example, urine from a subject to be tested having or suspected to have at least one disease or symptom selected from the group consisting of hypertension, primary aldosteronism, diabetes, obesity and a renal disease.

The present application also relates to an invention

of a method for testing blood aldosterone concentration, comprising the measuring method of the present invention described above. MR activity measurable by the measuring method of the present invention described above has a linear correlation with blood aldosterone concentration, and hence blood aldosterone concentration can be estimated or predicted from MR activity obtained by the measuring method of the present invention. Subjects also include patients exhibiting no linear relationship between MR activity and blood aldosterone concentration, such as patients receiving pharmacotherapy with a MR inhibitor and patients having excessive cortisol, and hence the method for testing blood aldosterone concentration according to the present invention is preferably performed in combination with another test and investigation for a medication status.

Herein, the blood aldosterone concentration encompasses plasma aldosterone concentration and serum aldosterone concentration.

The measuring method of the present invention encompasses a method comprising a step of recovering extracellular vesicles from urine collected from a subject to be tested, and a method free of the step. A method of recovering extracellular vesicles from urine is not particularly limited. Known recovery methods may be used. Examples of the recovery methods include ultracentrifugation, polymeric precipitation, immunoprecipitation, gel filtration, and combinations thereof.

The measuring method of the present invention comprises a step of quantifying an ENaC subunit. Quantification methods for the ENaC subunit and other proteins are not particularly limited. Known protein quantification methods may be used. Examples of the measuring method include Western blot and ELISA.

The present invention is described below in more detail with reference to Examples, but the present invention is not limited thereto.

### EXAMPLES

### 1. Materials and Methods

### 1.1 Subjects

Enrolled subjects were 18 patients hospitalized in Osaka University Hospital from November 2019 to December 2020 and measured for plasma aldosterone concentration (PAC) and plasma renin activity (PRA) to assess aldosterone production. The purposes of hospitalization of the subjects included suspected primary aldosteronism (PA), hormonal screening for identifying a function of an adrenal tumor, sampling of an adrenal vein from a PA patient, and optimization of glycemic control in diabetes and essential hypertension (EH) patients. All the subjects were informed and consented to participate in the study. The characteristics of the subjects are shown in Table 1. The subjects were given hospital meals containing less than 8 g of salt per day. The first void of urine in the early morning was collected from the patients and was frozen until treatment for extracellular vesicle (EV) extraction. Two samples that had not been completely soluble in an EV extraction process were excluded and 16 samples were analyzed. PAC, PRA, and other laboratory data were collected from medical records of the patients. This study was approved by the ethics committee of Osaka University and was conducted in accordance with the Declaration of Helsinki.

1.2 Urinary Extracellular Vesicle (uEV) Extraction

The method of Fernandez-Llama et al. (Kidney International, 77, 736-742, 2010) and the method of Merchant et al. (Nature Reviews: Nephrology, 13, 731-749, 2017) were modified to extract uEVs. First, the frozen urine samples were thawed in a water bath at 30° C. After the samples were melted, 20 mL of the samples were promptly transferred to different tubes, and protease inhibitors (0.67 mL of 100 mM NaN₃, 1 mL of PMSF (2.5 mg/mL in isopropyl alcohol), and 2 μL of leupeptin (10 mg/mL)) were added thereto. The samples were centrifuged at 3,000 g at 4° C. for 10 minutes, and the supernatant was recovered in another tube in order to remove debris. Then, the supernatant was centrifuged at 17,000 g at 4°° C. for 10 minutes. The supernatant was transferred to another tube (SN1), and the pellet (P1) was dissolved in 50 μL of an isolation solution containing 200 mg/mL DTT. The isolation solution contained 10 mM triethanolamine and 250 mM sucrose in H20 and had a pH of 7.6. After dissolved, P1 was incubated at 37° C. for 10 minutes. Subsequently, 1 mL of an isolation solution without DTT was added to P1 and the mixture was centrifuged at 17, 000 g at 4° C. for 10 minutes. The supernatant (SN2) was mixed with SN1. Finally, SN1+SN2 was ultracentrifuged at 200,000 g at 4°° C. for 77 minutes with a 70Ti rotor (Beckman Coulter, Inc., Brea, CA), and the supernatant was discarded. The pellets were dried in a spin drier at 40° C. for 30 minutes. The dried pellets were used for Western blotting.

### 1.3 Western Blotting

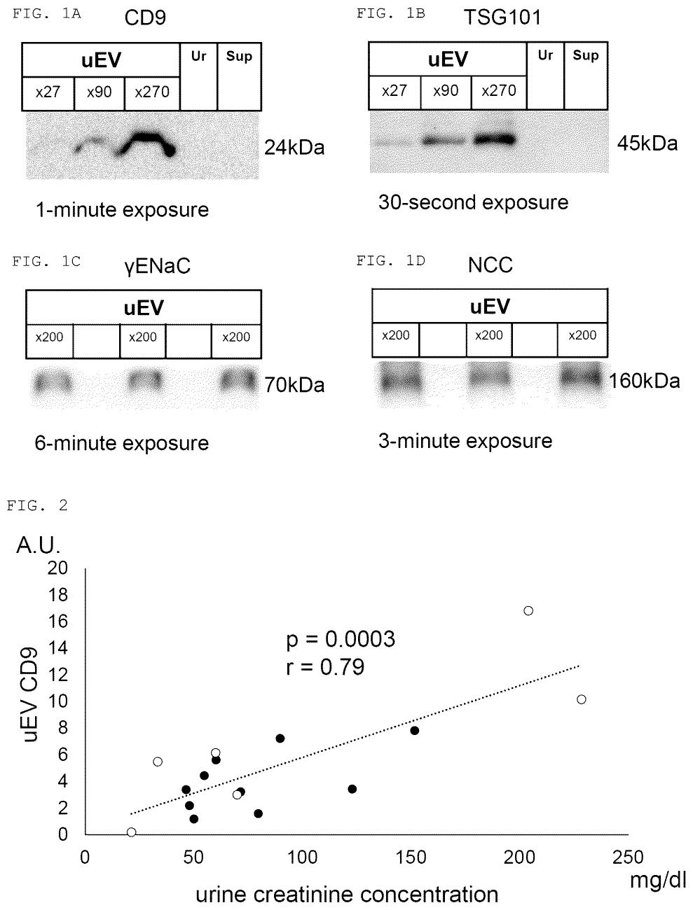

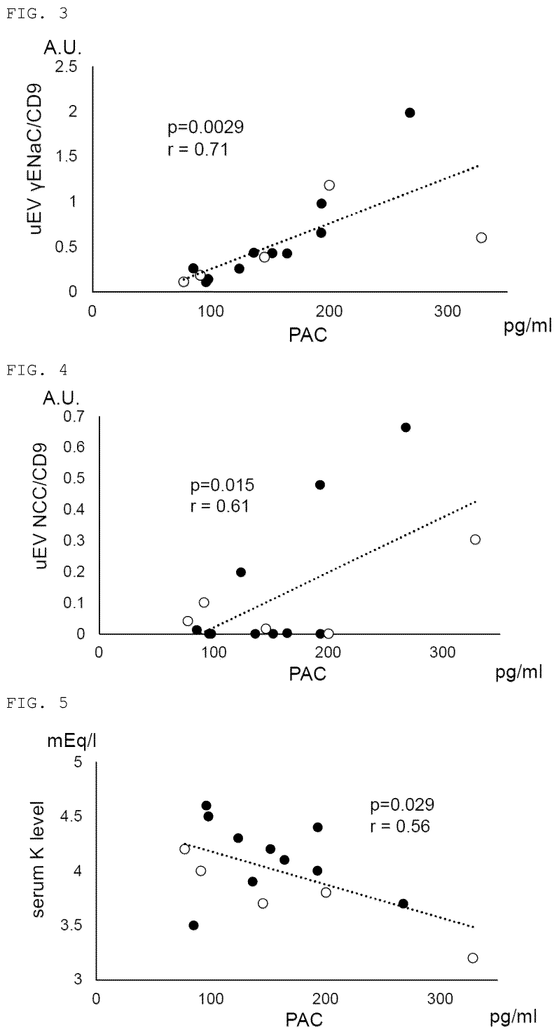

Protein immunoassay was performed by a common Western blotting method. In brief, the sample pellets were dissolved in 100 μL of Laemmli SDS sample buffer (FUJIFILM Wako Pure Chemical Corporation) containing 2-mercaptoethanol (ME). The final sample was a 200-fold concentrated liquid of the original urine (20 mL to 100 μL). The 200-fold concentrated liquid was used for measurement, but for checking the recovery of extracellular vesicles, 27-fold, 90-fold, and 270-fold concentrated liquids were used beforehand to confirm that CD9 and TSG101 were able to be extracted (FIG. 1).

Next, the dissolved samples were incubated at 95° C. for 5 minutes, vortexed, and centrifuged. The SDS samples were electrophoresed and transferred to PVDF membranes. The PVDF membranes were blocked with 5% skimmed milk and washed three times with Tris-buffered saline containing Tween 20 (TBS-T). Primary antibodies were incubated with the membranes at 4° C. overnight. Antibodies against CD9, TSG101, γENaC, and NCC were purchased from Santa Cruz Biotechnology Inc. (#SC-13118), Abcam Plc. (#ab125011), StressMarq Biosciences Inc. (Victoria, BC, Canada, #SPC-405), and Merck KGAA (#AB3553), respectively. Anti-mouse IgG (NA931) and anti-rabbit IgG (NA934) from Cytiva (Marlborough, MA) were used as peroxidase-conjugated secondary antibodies. Chemiluminescence was performed with Pierce ECL Plus (Thermo Fisher Scientific KK).

### 1.4 Preparation of Standard Proteins

Standard proteins of human CD9, TSG101, and NCC were prepared from pooled urine from healthy volunteers. The pooled urine was treated according to the above-mentioned uEV extraction protocol, and proteins in the extracted uEVs were used as standard proteins.

A protein produced by HEK293T cells transfected with an expression vector containing a human γENaC gene was used as a human γENaC standard protein. The plasmid was provided by Dr. Christie Thomas (Addgene Plasmid #83428) (Raikwar N S & Thomas C P, American Journal of Physiology: Renal Physiology, 294, F1157-F1165, 2008). Cells were collected and lysed with a lysis buffer containing 20 mM Tris/HCl PH 7.5, 1.0% Triton, 150 mM NaCl, and 1.0 mM EGTA. Then, the lysate was centrifuged at 16,500 g at 4° C. for 10 minutes. The supernatant was mixed with a 4×SDS sample buffer containing 2-ME, and Western blot analysis was performed.

### 1.5 Quantification of Proteins

Quantification of proteins was performed by

modifying a method reported previously (Heidebrecht F et al., Journal of Immunological Methods, 345, 40-48, 2009). First, the PVDF membrane, to which serially diluted standard proteins and uEV samples had been transferred, and which had been treated as described above, was imaged with Molecular Imager ChemiDoc XRS+System (Bio-Rad Laboratories Inc.). This system includes a cooled and calibrated CCD sensor with a linear response curve. Chemiluminescence images were obtained by using Pierce ECL Plus reagent with appropriate exposure time, resulting in clear band images without oversaturation. The images were captured in the native format using the standard software of the system, and exported to 16-bit TIFF format. Then, band densitometry was performed by using the public domain ImageJ program (developed at the National Institutes of Healthcare) with background density subtraction, and standard curves were created with serial standard protein band densities. Our standard curves were very well fitted and were thus formed of four points through use of quadratic approximation.

### 1.6 Endocrinological Evaluation

A plasma aldosterone concentration (PAC) was measured with Accuraseed Aldosterone Kit (FUJIFILM Wako Pure Chemical Corporation). PRA was measured with a renin activity kit (Yamasa Corporation, Choshi, Japan). PAC and PRA were measured in the morning under hospitalized conditions. PRA values of less than 0.2 ng/mL/h were regarded as 0.1 ng/mL/h. Only the PAC values after spironolactone treatment were measured in outpatient care in the morning. PA patients were diagnosed in accordance with the consensus statement 2016 of The Japan Endocrine Society (Naruse et al.). In summary, a patient having a ratio between aldosterone and renin of more than 200 pg/mL/ng/mL/h was considered as screening positive and received a confirmatory test. When a positive result was obtained in one of the confirmatory tests including a captopril tolerance test or a saline tolerance test, the patient was diagnosed as PA.

### 1.7 Evaluation of Creatinine in Urine

Urine creatinine concentration was measured by using colorimetry according to a manufacturer's protocol (ab65340; Abcam Plc.). The same frozen urine sample as that used in the uEV extraction was thawed and analyzed (Wolley MJ et al., Journal of the American Society of Nephrology, 28, 56-63.).

### 1.8 Statistical Analysis

The clinical features of the patients are presented as means and ranges (minimum-maximum). Differences between groups were analyzed with the Wilcoxon test. Correlations between two groups were tested by single regression analysis. P<0.05 was considered to indicate statistical significance. For all analyses, JMP Pro software version 15.1.0.0 for Windows (SAS Institute, Cary, NC) was used.

### 2. Results

### 2.1 Features of Patients

Baseline clinical characteristics of 16 analyzed patients are shown in Table 1. The 16 analyzed patients included 5 PA patients having a plasma aldosterone concentration of less than 160 pg/mL (nPA), 4 PA patients having a plasma aldosterone concentration of 160 pg/mL or more (hPA), 1 PA patient accompanied with Cushing syndrome (PA with CS), 5 patients with essential hypertension (EH), and 1 patient having normal blood pressure and adrenal mass without any hormone function (nonPAnonEH). No patient took any MR antagonist.

TABLE 1

| Features | nPA | | hPA | | PA with CS | EH | | NonPAnonEH |
|---|---|---|---|---|---|---|---|---|
| Number of patients | 5 | | 4 | | 1 | 5 | | 1 |
| Sex | | | | | | | | |
| Female | 3 | | 2 | | 1 | 0 | | 0 |
| Male | 2 | | 2 | | 0 | 5 | | 1 |
| Age | 67 | (50-74) | 45 | (38-57) | 46 | 58 | (45-69) | 75 |
| Blood pressure (mmHg) | | | | | | | | |
| Diastolic | 137 | (125-147) | 128 | (110-150) | 93 | 121 | (94-146) | 127 |
| Systolic | 81 | (63-94) | 86 | (63-111) | 52 | 72 | (53-90) | 75 |
| PAC (pg/mL) | 107 | (85.1-145.5) | 222 | (164.2-328.5) | 66.2 | 167 | (97.7-267.8) | 95.9 |
| PRA (ng/mL/h) | 0.16 | (0.1-0.3) | 0.3 | (0.2-0.4) | 0.3 | 10.2 | (0.9-38.8) | 1.3 |
| ARR (pg/mL/ng/mL/h) | 744.0 | (454.3-914.0) | 799.6 | (410.5-1,001.5) | 220.7 | 65.8 | (3.2-168.8) | 73.8 |
| Number of antihypertensive | 1.2 | (0-3) | 1 | (1-1) | 0 | 1.6 | (1-2) | 0 |
| classes | | | | | | | | |
| Number of patients with | | | | | | | | |
| antihypertensives | | | | | | | | |
| With CCB | 4 | | 4 | | 0 | 3 | | 0 |
| With ARB | 0 | | 0 | | 0 | 3 | | 0 |
| With α blocker | 1 | | 0 | | 0 | 2 | | 0 |
| With β blocker | 1 | | 0 | | 0 | 0 | | 0 |
| Serum K (mEq/L) | 3.9 | (3.5-4.2) | 3.8 | (3.2-4.1) | 3.9 | 4.2 | (3.7-4.5) | 4.6 |

Data in Table 1 is presented as the number of patients or average (minimum-maximum). PAC: plasma aldosterone concentration; PRA: plasma renin activity; ARR: aldosterone-to-renin ratio (PAC/PRA); ARB: angiotensin II receptor blocker; CCB: calcium channel blocker.

### 2.2 Confirmation of uEV Extraction

uEVs were extracted and checked for expression of CD9 as a membrane-bound marker and TSG101 as an intravesicular marker of uEVs (FIG. 1). Both CD9 and TSG101 were observed in concentrated uEV fractions (x27, x90, x270), but not in urine itself or the supernatant after ultracentrifugation. γENaC and NCC proteins were detected in concentrated uEV fractions (x200). To collect abundant uEVs, the first void of urine in the early morning, a concentrated sample, was used and DTT was employed in an extraction process. A γENaC band was located at about 70 kDa, which indicated a cleaved form (active form) of γENaC (Pisitkun T et al., PNAS, 101, 13368-13373, 2004; Frindt G et al., Journal of General Physiology, 147, 217-227, 2016).

2.3 CD9 Protein Quantity in uEVs Correlates with Urine Creatinine Quantity

Salih et al. found an excellent correlation between urine creatinine and CD9 in uEVs (Salih M et al., American Journal of Physiology: Renal Physiology, 310, F796-F801, 2016). To confirm the significance of CD9 in uEVs, Western blotting was performed to measure the intensity of a CD9 band in uEVs. The value of CD9 was calculated based on the standard CD9 protein. The CD9 quantity in uEVs and urine creatinine concentration of the 16 samples exhibited a significant correlation (FIG. 2). The correlation coefficient was 0.79. The line approximately crossed the origin of the graph. The graph includes all the 16 samples. There was no clear difference between sexes.

2.4 γENaC/CD9 in uEVs has Better Correlation with Plasma Aldosterone Concentration (PAC) than NCC/CD9 in uEVs or Serum Potassium Concentration

γENaC and NCC in uEVs were measured in 15 patients including nPA, hPA, EH, and nonPAnonEH patients (Table 1) by quantitative densitometric analysis of the Western blotting results. One patient with a combination of PA and adrenal Cushing syndrome (CS) (“PA with CS” in Table 1) was excluded from the analysis. CS is known to cause excess cortisol secretion and can activate MR by a spill-over effect (Palermo et al., 1996). Accordingly, one sample of “PA with CS” was determined to be excluded, and the remaining 15 samples were analyzed. Each protein quantity was calculated by using the standard curve created by serial dilution of the prepared standard protein.

Aldosterone is a physiological ligand of the MR. Accordingly, an indicator (biomarker) of MR activity is expected to correlate with plasma aldosterone concentration (PAC). A protein quantity of γENaC in uEVs, a protein quantity of NCC (NaCl cotransporter) in uEVs, serum potassium concentration, and plasma renin activity (PRA) were investigated for potential availability as biomarkers. For the protein quantity of γENaC in uEVs and the protein quantity of NCC in uEVs, values normalized with CD9 were used. The values normalized with CD9 are a ratio of the protein quantity of γENaC to that of CD9 in uEVs (γENaC/CD9) and a ratio of the protein quantity of NCC to that of CD9 in uEVs (NCC/CD9).

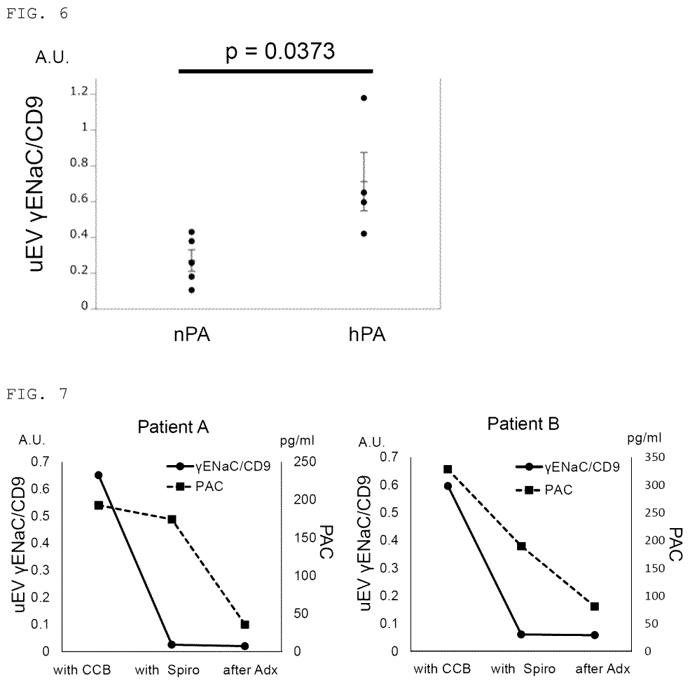

Plasma aldosterone concentration (PAC) and γENaC/CD9 had a quite strong correlation. The correlation coefficient was 0.71 (FIG. 3). PAC and NCC/CD9 had a slight correlation. The correlation coefficient was 0.61 (FIG. 4). Serum potassium concentration exhibited a negative correlation with PAC (FIG. 5). However, the correlation coefficient between serum potassium concentration and PAC was smaller than the correlation coefficients between γENaC/CD9 and PAC and between NCC/CD9 and PAC, indicating a weaker correlation. PRA exhibited no correlation with PAC. In summary, γENaC/CD9most strongly correlated with PAC. No clear difference was observed between sexes.

2.5 γENaC/CD9 in uEVs is higher in hPA Patients than in nPA Patients

Autonomous aldosterone secretion directly induces organ damage via MR activation. Accordingly, patients with primary aldosteronism (PA) have an incidence of cardiovascular and cerebrovascular (CCV) events higher than that of patients with essential hypertension (EH) (Catena C et al., Archives of Internal Medicine, 168, 80-85, 2008.; Mulatero P et al., Journal of Clinical Endocrinology and Metabolism, 98, 4826-4833., 2013; Monticone S et al., Lancet: Diabetes and Endocrinology, 6, 41-50, 2018.; and Ohno Y et al., Hypertension, 71, 530-537, 2018.). The inventors previously reported that the prevalence of CCV events is higher in PA patients with hPA (PAC of 160 pg/mL or more) than in those with nPA (PAC of less than 160 pg/mL) (Murata M et al., Journal of Hypertension, 35, 1079-1085, 2017). γENaC/CD9 was measured in five nPA patients and four hPA patients (Table 1). The hPA patients exhibited γENaC/CD9 higher than that of the nPA patients (FIG. 6). The results provided further evidence that γENaC/CD9 is a biomarker of MR activity and can be an indicator for assessing the risk of CCV events.

2.6 γENaC/CD9 in uEVs is reduced by Treatment for Primary Aldosteronism

MR activity is reduced by MR antagonist therapy and surgical adrenalectomy (Adx). γENaC/CD9 was measured in two patients during PA treatment. The two PA patients were hospitalized for Adx. Patient A took spironolactone at 200 mg/day, and patient B took spironolactone at 50 mg/day. The patients underwent Adx and used no MR antagonist after Adx. Urine samples were collected three times from those patients. The collection of the urine samples was performed at three time points: during treatment only with a calcium channel blocker as an antihypertensive (“with CCB” in FIG. 7); during treatment with spironolactone before Adx (“with Spiro” in FIG. 7); and after Adx (“after Adx” in FIG. 7). Spironolactone, an MR antagonist, dramatically reduced γNaC/CD9, but had only a small effect on plasma aldosterone concentration (PAC) because aldosterone was autonomously produced in both patients (FIG. 7). Surgical adrenalectomy (Adx) effectively reduced both PAC and γENaC/CD9 in both patients (FIG. 7). In summary, MR activity is reduced by MR antagonist therapy and surgical adrenalectomy (Adx), and γENaC/CD9 decreased during MR antagonist therapy and after surgical adrenalectomy (Adx). Thus γENaC/CD9 was suitable as an MR activity indicator. Those results demonstrate that γENaC/CD9 reflects MR activity during PA treatment.

### INDUSTRIAL APPLICABILITY

The present invention provides a noninvasive and convenient method of measuring MR activity and enables prevention of onset and therapeutic intervention suitable for individual patients and potential patients of cerebrovascular and cardiovascular diseases.

1. A method for measuring mineralocorticoid receptor activity using an indicator, wherein the indicator is a protein quantity of an epithelial sodium channel subunit in urinary extracellular vesicles.

2. The method according to claim 1, wherein the indicator is a ratio of a protein quantity of an epithelial sodium channel subunit to a protein quantity of an internal control protein in extracellular vesicles.

3. The method according to claim 2, wherein the internal control protein of the extracellular vesicles is a tetraspanin.

4. The method according to claim 3, wherein the tetraspanin is CD9.

5. The method according to claim 1, wherein the subunit comprises at least one selected from the group consisting of an α subunit, a β subunit and a γ subunit.

6. The method according to claim 5, wherein the subunit is the γ subunit.

7. The method according to claim 1, wherein the method comprises using, as a sample, urine of a subject to be tested having or suspected to have at least one disease or symptom selected from the group consisting of hypertension, primary aldosteronism, diabetes, obesity and a renal disease.

8. The method according to claim 1, wherein the mineralocorticoid receptor activity is mineralocorticoid receptor activity in a tubular epithelial cell.

9. A method for testing a disease associated with mineralocorticoid receptor activation comprising the method of claim 1.

10. The method according to claim 9, wherein the disease associated with mineralocorticoid receptor activation is mineralocorticoid receptor-associated hypertension.

11. A method for testing a risk of onset of cerebrovascular and cardiovascular diseases comprising the method of claim 1.

12. The method according to claim 11, wherein the cerebrovascular and cardiovascular diseases comprise an arteriosclerotic disease.

13. A method for determining a therapeutic effect on a disease associated with mineralocorticoid receptor activation comprising the method of claim 1.

14. The method according to claim 13, wherein the disease associated with mineralocorticoid receptor activation is primary aldosteronism.

15. A method for testing blood aldosterone concentration comprising the method of claim 1.
